(19) United States
(12) Patent Application Publication
(10) Pub. No.: US 2004/0076586 A1
(43) Pub. Date: Apr. 22, 2004

(54) Compositions and methods for delivering pharmaceutically active agents using nanoparticulates

(76) Inventors: Reinhard Koening, Washington, DC (US); Taffy J. Williams, Lansdale, PA (US)

Correspondence Address: LAHIVE & COCKFIELD, LLP., 28 STATE STREET, BOSTON, MA, 02109, US

(21) Appl. No.: 10/402,683

(22) Filed: Mar. 28, 2003

Related U.S. Application Data
(60) Provisional application No. 60/368,385, filed on Mar. 28, 2002.

(51) Int. Cl. A61L 9/04; A61K 9/14

(52) U.S. Cl. 424/46

(57) ABSTRACT

The present invention is directed to methods and pharmaceutical compositions, e.g., nanoparticulate drug delivery vehicles, for delivering pharmaceutically active agents to tissues and areas containing mononuclear phagocytes e.g., macrophages in order to treat inflammatory diseases or disorders, e.g., a mononuclear phagocyte-associated disease or disorder, infected biological areas or tissue, injured tissue, or disease tissue. The inflamed, infected, injured, or diseased tissue can be accessible through the blood stream, using a nanoparticulate drug delivery vehicle injected into vascular beds (such as for example arterial and venous beds). Alternatively, the nanoparticulate drug delivery vehicle and pharmaceutically active agent of the invention may be administered locally, to treat specific areas or tissues, e.g., inflamed, infected, injured, or diseased tissue. In one embodiment, the nanoparticulate drug delivery vehicle is formulated as a contrast agent. Accordingly, imaging of the target area or tissue may be carried out prior to, during, or after administration of the nanoparticulate drug delivery vehicle.

### RELATED APPLICATIONS

This application claims priority to U.S. Provisional Application No. 60/368,385, filed on Mar. 28, 2002, the contents of which are incorporated herein by reference.

### BACKGROUND OF THE INVENTION

The inflammatory response serves the purpose of eliminating harmful agents from the body. There is a wide range of pathogenic insults that can initiate an inflammatory response including infection, allergens, autoimmune stimuli, immune response to transplanted tissue, noxious chemicals, and toxins, ischemia/reperfusion, hypoxia, mechanical and thermal trauma. Inflammation normally is a very localized action which serves in expulsion, attenuation by dilution, and isolation of the damaging agent and injured tissue. The body's response becomes an agent of disease when it results in inappropriate injury to host tissues in the process of eliminating the targeted agent, or responding to a traumatic insult.

As examples, inflammation is a component of pathogenesis in several vascular diseases or injuries. Examples include: ischemia/reperfusion injury (N. G. Frangogiannis et al., in Myocardial Ischemia: Mechanisms, Reperfusion, Protection, M. Karmazyn, ed., Birkhuser Verlag (1996) at 236-284; H. S. Sharma et al., (1987) Med. of Inflamm., 6, 175), atherosclerosis (R. Ross, (1993) Nature, 362, 801), inflammatory aortic aneurysms (N. Girardi et al., (1997) Ann. Thor. Surg., 64, 251; D. I. Walker et al., Brit. J. (1972) Surg., 59, 609; R. L. Pennell et al., (1985) J Vase. Surg., 2, 859), and restenosis following balloon angioplasty (see, R. Ross cited above). The cells involved with inflammation include leukocytes (i.e., the immune system cells—neutrophils, eosinophils, lymphocytes, monocytes, basophils, macrophages, dendritic cells, and mast cells), the vascular endothelium, vascular smooth muscle cells, fibroblasts, and myocytes.

Macrophages are white blood cells that are dedicated to the removal of foreign materials within body tissues. It has been shown that macrophages have the ability to migrate to areas of inflammation and deposits of foreign material, such as vascular plaques. The vascular system of the body, for example, can become damaged due to a build-up of plaque. Such a build-up of plaque can lead to stroke, ischemia, poor circulation or heart attacks. However, if the cells in the region of the plaque can be destroyed, the active plaque may resolve and decrease the danger of rupture with consecutive thrombosis or embolism, leading to myocardial infarction.

Therefore, the ability to specifically deliver drugs to macrophages would be important in the treatment of various diseases and disorders, including inflammatory diseases, immune diseases, and diseases or disorders related to vascular plaque.

### SUMMARY OF THE INVENTION

The present invention is directed to methods and pharmaceutical compositions, e.g., nanoparticulate drug delivery vehicles, for delivering pharmaceutically active agents to sites of inflammation, infection, injury or disease in a subject. In one embodiment, the pharmaceutically active agents are delivered to tissues and areas containing mononuclear phagocytes, e.g., macrophages. The tissue can be accessible through the blood stream, using a nanoparticulate drug delivery vehicle injected into vascular beds (such as for example arterial and venous beds). Alternatively, the nanoparticulate drug delivery vehicle and pharmaceutically active agent of the invention may be administered locally, to treat specific areas or tissues, e.g., infected, inflamed, diseased or injured tissue. Once delivered, the pharmaceutically active agent can be used for treatment of inflammatory diseases or disorders, infectious diseases or disorders, tumorigenic diseases or disorders, infected tissue, injured tissue or diseased tissue. In one embodiment, the pharmaceutically active agent can be used for treatment of mononuclear phagocyte-associated diseases or disorders. In another embodiment, the nanoparticulate drug delivery vehicle is formulated as a contrast agent. Accordingly, imaging of the target area or tissue may be carried out prior to, during, or after administration of the nanoparticulate drug delivery vehicle.

Therefore, in one aspect, the present invention is directed to pharmaceutical compositions comprising a nanoparticulate drug delivery vehicle and a pharmaceutically active agent. In one embodiment, the nanoparticulate drug delivery vehicle is non-water soluble. In another embodiment, the nanoparticulate drug delivery vehicle is PH-50. In a further embodiment, the mean particle size of the nanoparticulate drug delivery vehicle is from about 20 nanometers to about 750 nanometers, from about 200 nanometers to about 400 nanometers, or more preferably is about 300 nanometers. In a particularly preferred embodiment, the mean particle size of said nanoparticulate drug delivery vehicle is of a size sufficient to be taken up by mononuclear phagocytes, e.g., macrophages. In another aspect of the invention, the nanoparticulate drug delivery vehicle is formulated as a contrast agent, thereby allowing imaging of tissues and vessels either prior to, during, or after treatment with the pharmaceutical compositions of the invention.

In one embodiment of the invention, the nanoparticulate drug delivery vehicle is enzymatically degradable. In another embodiment, the nanoparticulate drug delivery vehicle and the pharmaceutically active agent are conjugated, e.g., covalently or non-covalenty conjugated. In a further embodiment, the pharmaceutically active agent coats the surface of the nanoparticulate drug delivery vehicle. In yet another embodiment, the nanoparticulate drug delivery vehicle contains a hollow core and the pharmaceutically active agent is encapsulated by the nanoparticulate. For example, the nanoparticulate may be a liposome. In still another embodiment, the pharmaceutically active substance is a prodrug. In one embodiment, the prodrug itself may be formulated as a nanoparticulate. In another embodiment, the prodrug is metabolically converted to an active substance upon uptake by a mononuclear phagocyte, e.g., a macrophage, or upon administration to a subject. In still another embodiment, the pharmaceutically active agent is a radiopharmaceutical agent. In yet another embodiment, the pharmaceutically active agent is a sustained release agent.

In a further aspect, the present invention provides methods for treatment of inflammatory diseases or disorders, infectious diseases or disorders, infected areas or tissue, injured tissue or diseased tissue in a subject comprising administering to said subject an effective amount of a nanoparticulate drug delivery vehicle and a pharmaceutically active agent, thereby treating said inflammatory diseases or disorders, infectious diseases or disorders, infected areas or tissue, injured tissue or diseased tissue. In another aspect of the invention, the present invention provides methods for treatment of a mononuclear phagocyte-associated disease or disorder in a subject comprising administering to said subject an effective amount of a nanoparticulate drug delivery vehicle and a pharmaceutically active agent, thereby treating said mononuclear phagocyte-associated disease or disorder. In one embodiment, the composition is administered intravenously. In another embodiment, the composition is locally injected at a site of infection or inflammation.

In another aspect, the invention provides methods, e.g., in vivo methods, for treating or removing plaque accumulation in a blood vessel of a subject comprising administering to said subject an effective amount of a composition comprising a nanoparticulate and a pharmaceutically active agent, thereby treating or removing plaque accumulation in a blood vessel of a subject.

In yet another aspect, the invention provides methods, e.g., in vivo methods, for treating a tumorigenic disease or disorder in a subject comprising administering to said subject an effective amount of a composition comprising a nanoparticulate drug delivery vehicle and a pharmaceutically active agent, thereby treating said tumorigenic disease or disorder.

### BRIEF DESCRIPTION OF THE DRAWINGS

FIG. 1 depicts the chemical structure of PH-50.

FIGS. 2A and 2B depict an image of a rabbit heart before PH-50 injection (FIG. 2A) and a rabbit heart after PH-50 injection (FIG. 2B).

FIGS. 3A and 3B depict an image of a rabbit liver before (FIG. 3A) and after (FIG. 3B) PH-50 contrast agent injection, respectively.

FIGS. 4A and 4B are images of a CT scan of a rabbit kidney after injection of PH-50 contrast agent.

FIG. 5 is an image of nanoparticulates taken up in vascular plaque present in rabbit heart.

FIG. 6 is an image of nanoparticulates taken up in vascular plaque present in rabbit myocardial artery.

### DETAILED DESCRIPTION OF THE INVENTION

The present invention provides, at least in part, methods and compositions for the delivery of pharmaceutically active agents to sites of inflammation, infection, injury, or disease in a subject. In one embodiment, the pharmaceutically active agents are delivered to tissues or areas of the body which contain mononuclear phagocytes, e.g., macrophages, e.g., areas which contain accumulated mononuclear phagocytes, e.g., macrophages, such as, for example, vascular plaques, lymph nodes, liver, spleen, and areas of diseased, infected, injured, or inflamed tissue. In another aspect, the invention provides methods and compositions for delivery of a pharmaceutically active agent and imaging the target tissue or area, either prior to, during, or after the delivery of the pharmaceutically active agent. Therefore, the present invention provides methods and compositions for treating, identifying, and/or diagnosing inflammatory diseases or disorders, infectious diseases or disorders, tumorigenic diseases or disorders, injured tissue, e.g., tissue injured or traumatized by medical procedures such as, for example, angioplasty, reperfusion, or organ transplantation, or diseased tissue. In another embodiment, the present invention provides methods and compositions for treating, identifying, or diagnosing mononuclear phagocyte-associated diseases or disorders. The present invention is not limited to a particular tissue or area of the body which may be targeted for delivery and/or imaging using the methods and compositions of the invention.

It has been discovered that particulates of a certain size, e.g., nanoparticulates in the size range of 5-2,000 nanometers, for example, are phagocytized by mononuclear phagocytes, e.g., macrophages. Accordingly, these nanoparticulates can serve as a means for delivering agents, e.g., pharmaceutically active agents, selectively to inflamed, infected, injured or diseases tissue or to mononuclear phagocytes, e.g., macrophages, for example, areas of accumulated mononuclear phagocytes, e.g., macrophages including diseased or inflamed tissues, or to vasculature containing plaque, lymph nodes, liver, and spleen. In one embodiment, certain pathogenic diseases, e.g., tuberculosis, wherein the infective agent, e.g., a bacterial, viral, or fungal pathogen, is contained within the mononuclear phagocyte, e.g., macrophage, may be targeted with the agents of the present invention. The ability to deliver pharmaceutically active agents to mononuclear phagocytes, e.g., macrophages may also have applications for gene therapy. The nanoparticulates of the invention which may be used to deliver pharmaceutically active agents are referred to herein as nanoparticulate drug delivery vehicles.

A nanoparticulate drug delivery vehicle of the present invention may be formulated as contrast agent. The term “contrast agent,” as used herein, includes any substance that can be introduced, e.g., injected, into a structure, e.g., an organ, tissue, blood vessel, blood pool, or plaque, and, because of the difference in the absorption of detection medium, e.g., x-rays, radiowaves, soundwaves or the like, between the contrast agent and the structure, allow for detection, visualization, or enhanced visualization, e.g., radiographic or sonographic visualization, of the structure, e.g., the organ, tissue, blood vessel, blood pool, or plaque. When the nanoparticulate drug delivery vehicles of the invention are formulated as contrast agents, because the nanoparticulate drug delivery vehicles of the invention are preferably taken up by mononuclear phagocytes, e.g., macrophages, e.g., activated macrophages, visualization of the tissue, vascular bed, or organ containing the mononuclear phagocytes is possible using routine imaging technology, e.g., by x-ray imaging, ultrasonagraphy, computed tomography (CT), computed tomography angiography (CTA), electron beam (EBT), magnetic resonance imaging (MRI), magnetic resonance angiography (MRA), positron emission tomography, and other imaging technologies.

When administered to a subject, the preferred nanoparticulate drug delivery vehicle, e.g., PH-50, remains substantially confined to the intravascular space and therefore does not permeate to the interstitial space or extrastitial fluids, thus facilitating the imaging and/or treatment of mononuclear phagocyte-associated diseases or disorders. Furthermore, preferred nanoparticulate drug delivery vehicle of the invention, e.g., PH-50, is excreted from the body via the hepatic system rather than the renal system, and therefore remains in the body for a longer period of time than agents which are excreted via the renal system. Furthermore, excretion via the hepatic system permits usage of the preferred nanoparticulate drug delivery vehicles of the invention, e.g., PH-50, in patients with renal insufficiency, and also permits imaging and/or treatment of diseases of the renal system including the abdominal aorta and renal arteries, e.g., hypertension, diabetes, or cancer, e.g., kidney tumors. Furthermore, it is believed that such nanoparticulate drug delivery vehicle, e.g., PH-50, will not cause renal system damage.

Certain embodiments of the invention feature nanoparticulate drug delivery vehicles, e.g., PH-50, which remain in the vascular structures for an extended period of time at functionally active concentrations with a half-life of about 30-60 minutes until the contrast agent is metabolized by the liver. Because of the extended time period in which the nanoparticulates remain in the vascular structures, the time-period for treatment of inflammatory diseases or disorders, infectious diseases or disorders, infected areas or tissue, tumorigenic diseases or disorders, injured tissue, or diseased tissue, e.g., mononuclear phagocyte-associated diseases or disorders, e.g., treatment of vascular plaque or other inflammatory diseases or disorders, infectious diseases or disorders, infected areas or tissue, tumorigenic diseases or disorders, injured tissue, or diseased tissue, is extended, thereby increasing the efficacy of the treatment.

“Treatment”, or “treating” as used herein, is defined as the application or administration of a pharmaceutically active agent to a patient who has an inflammatory disease or disorder, an infectious disease or disorder, infected areas or tissue, a tumorigenic disease or disorder, injured tissue, or diseased tissue, e.g., a mononuclear phagocyte-associated disease or disorder, a symptom of an inflammatory disease or disorder, an infectious disease or disorder, infected areas or tissue, a tumorigenic disease or disorder, injured tissue, or diseased tissue, e.g., a mononuclear phagocyte-associated disease or disorder, or a predisposition toward an inflammatory disease or disorder, an infectious disease or disorder, infected areas or tissue, a tumorigenic disease or disorder, injured tissue, or diseased tissue, e.g., a mononuclear phagocyte-associated disease or disorder, with the purpose to cure, heal, alleviate, relieve, alter, remedy, ameliorate, improve or affect an inflammatory disease or disorder, an infectious disease or disorder, infected areas or tissue, a tumorigenic disease or disorder, injured tissue, or diseased tissue, the symptoms thereof, or the predisposition thereto.

As used herein, the term “pharmaceutically active agent” refers to any chemical substance, e.g., any drug or compound that is used in the treatment, cure, prevention, or diagnosis or a mononuclear phagocyte-associated disease or disorder or otherwise to enhance the physical or mental well-being of a subject, e.g., a mammal, e.g., a human. Examples of pharmaceutically active agents which are included in the present invention include, without limitation, small molecules, peptides, ribozymes, antisense oligonucleotides, short interfering RNA (siRNA), radiopharmaceutical agents, chemotherapeutic agents for parasitic infections and microbial diseases, anti-cancer agents, e.g., alkylating agents (e.g., nitrosoureas) and antimetabolites; nitrogen mustards, ethylenamines and methylmelamines; alkylsulfonates; folic acid analogs; pyrimidine analogs, purine analogs, vinca alkaloids, anti-inflammatory agents, e.g., phenylbutazone, indomethacin, naproxen, ibuprofen, flurbiprofen, diclofenac, dexamethasone, prednisone and prednisolone, histamine, bradykinin, kallidin and their respective agonists and antagonists, immune modulatory agents, anti-infective agents, anti-viral agents, lipid-lowering agents, cytokine modulating agents, anti-thrombogenic drugs, such as heparin or a heparin derivative, anti-proliferative drugs such as enoxaprin, angiopeptin, or antibodies, e.g., polyclonal antibodies or monoclonal antibodies, hirudin or acetylsalicylic acid (i.e., aspirin). In other embodiments, the drug is a naked nucleic acid or a nucleic acid incorporated into a viral vector. By naked nucleic acid is meant a uncoated single or double stranded DNA or RNA molecule. In another embodiment, the pharmaceutically active agent is non-water soluble. In still another embodiment, the drug is a prodrug which is metabolically converted into an active agent once administered to the subject or once taken up by a mononuclear phagocyte, e.g., a macrophage. In a further embodiment, the pharmaceutically active agent is a sustained-release agent.

As used herein, the term “mononuclear phagocyte” includes, for example, macrophages, monocytes, microglial cells, and dendritic cells. Mononuclear phagocytes, e.g., macrophages, are involved in all stages of immune responses. Mononuclear phagocytes, e.g., macrophages, play an important role in the phagocytosis (digestion) of foreign bodies, such as bacteria, viruses, protozoa, tumor cells, cell debris and the like, as well as the release of chemical substances, such as cytokines, growth factors and the like, that stimulate other cells of the immune system. Macrophages are relatively long-lived phagocytic cells of mammalian tissues, derived from blood monocytes. Macrophages are also involved in antigen presentation as well as tissue repair and wound healing. There are many types of macrophages, including aveolar and peritoneal macrophages, tissue macrophages (histiocytes), Kupffer cells of the liver and osteoclasts of the bone, all of which are within the scope of the invention. Macrophages may also further differentiate within chronic inflammatory lesions to epitheliod cells or may fuse to form foreign body giant cells (e.g., granulomas) or Langerhan giant cells.

Infectious diseases or disorders include any disease or disorder caused by or related to infection by a pathogen, e.g., a viral, bacterial, or fungal pathogen. Infected tissue includes any biological tissue which is infected by any pathogen, e.g., any viral, bacterial, or fungal pathogen. Infected tissue may, for example, be characterized by accumulation of mononuclear phagocytes, e.g., macrophages.

Inflammatory diseases or disorders are characterized by diseased, infected, or inflamed tissue and are usually associated with mononuclear phagocytes, e.g., macrophages. Examples of inflammatory diseases or disorders include, without limitation, asthma, chronic pulmonary inflammatory disease, rheumatoid spondylitis, ankylosing sponduylitis, osteoarthritis and gouty arthritis, multiple sclerosis, chronic granulomatous diseases such as tuberculosis, leprosy, sarcoidosis, silicosis and schistosomiasis, nephritis, amyloidosis, rheumatoid arthritis, chronic bronchitis, scleroderma, lupus, polymyositis, appendicitis, inflammatory bowel disease, ulcers, Sjorgen's syndrome, Reiter's syndrome, psoriasis, pelvic inflammatory disease, orbital inflammatory disease, thrombotic disease, and inappropriate allergic responses to environmental stimuli such as poison ivy, pollen, insect stings and certain foods, including atopic dermatitis and contact dermatitis. The inflammation can be due to pathological agents or can be due to physical, chemical or thermal trauma, or the trauma of medical procedures, such as organ, tissue or cell transplantation, angioplasty (PCTA), inflammation following ischemia/reperfusion, or grafting.

Inflammatory diseases or disorders also include immunological disorders such as autoimmune disorders (e.g., arthritis, graft rejection (e.g., allograft rejection), T cell disorders (e.g., AIDS), autoimmune diabetes), immune deficiency disorders, e.g., congenital X-linked infantile hypogammaglobulinemia, transient hypogammaglobulinemia, common variable immunodeficiency, selective IgA deficiency, chronic mucocutaneous candidiasis, or severe combined immunodeficiency.

Inflammatory diseases or disorders also include “mononuclear phagocyte-associated diseases or disorders,” which include any disease or disorder which results in the activation or accumulation of mononuclear phagocytes, e.g., macrophages. For example, a mononuclear phagocyte-associated disease or disorder includes disorders pertaining to, characterized by, causing, resulting from, or becoming affected by inflammation, infection, injury, or disease.

Also included within the meaning of the term “inflammatory disease or disorder” is any disease or disorder related to or resulting from plaque build-up, e.g., a vascular disease or disorder, including, without limitation, intravascular stenosis (narrowing) or occlusion (blockage) due to, for example, a build-up of plaque on the inner arterial walls, and diseases and disorders resulting therefrom. Vascular plaque, e.g., vulnerable plaques, contain mononuclear phagocytes, e.g., macrophages, e.g., activated macrophages, which accumulate on arterial walls. Examples of vascular diseases and disorders include, without limitation, atherosclerosis, CAD, MI, unstable angina, acute coronary syndrome, pulmonary embolism, transient ischemic attack, thrombosis (e.g., deep vein thrombosis, thrombotic occlusion and re-occlusion and peripheral vascular thrombosis), thromboembolism, e.g., venous thromboembolism, ischemia, stroke, peripheral vascular diseases, and transient ischemic attack. Also intended to be within the scope of the term vascular disease or disorder are thrombotic, or thromboembolic, events. The term “thrombotic or thromboembolic event” includes any disorder that involves a blockage or partial blockage of an artery or vein with a thrombosis. A thrombic or thrombolic event occurs when a clot forms and lodges within a blood vessel which may occur, for example, after a rupture of a vulnerable plaque.

The term “tumorigenic disease or disorder” includes a disease or disorder characterized by aberrantly regulated cell growth, proliferation, differentiation, adhesion, or migration, resulting in the production of or tendency to produce tumors. As used herein, a “tumor” includes a normal benign or malignant mass of tissue. Examples of tumorigenic diseases include cancer, e.g., carcinoma, sarcoma, lymphoma or leukemia, examples of which include, but are not limited to, ovarian, lung, breast, cervical, endometrial, uterine, hepatic, gastrointestinal, prostate, colorectal, and brain cancer.

As used herein, the term “plaque,” also commonly referred to as “atheromas,” refers to the substance which builds up on the interior surface of the vessel wall resulting in the narrowing of the vessel and is the common cause of CAD. Usually, plaque comprises fibrous connective tissue, lipids (i.e. fat) and cholesterol. Frequently deposits of calcium salts and other residual material may also be present. Plaque build-up results in the erosion of the vessel wall, diminished elasticity (e.g., stretchiness) of the vessel and eventual interference with blood flow. Blood clots may also form around the plaque deposits thus further interfering with blood flow. Plaque stability is classified into two categories based on the composition of the plaque. As used herein, the term “stable” or “inactive” plaques refers to those which are calcific or fibrous and do not present a risk of disruption or fragmentation. These types of plaques may cause anginal chest pain but rarely myocardial infarction in the subject. Alternatively, the term “vulnerable” or “active” plaque refers to those comprising a lipid pools covered with a thin fibrous cap. Within the fibrous cap is a dense infiltrate of smooth muscle cells, macrophages, and lymphocytes. Vulnerable plaques may not block arteries but may be ingrained in the arterial wall, so that they are undetectable and may be asymptomatic. Furthermore, vascular plaques are considered to be at a high risk of disruption. Disruption of the vulnerable plaque is a result of intrinsic and extrinsic factors, including biochemical, haemodynamic and biomechanical stresses resulting, for example, from blood flow, as well as inflammatory responses from such cells as, for example, mononuclear phagocytes, e.g., macrophages.

I. Nanoparticulates Used in the Methods and Compositions of the Invention

The term “nanoparticulate” or “nanoparticle” refers to a composition comprising particles having a mean diameter of preferably between about 5.0 nanometers and about 2.0 microns, typically between about 100 nanometers and 1.0 micron. In a preferred embodiment, the nanoparticulate contrast agent used in the methods of the invention has a mean particle size of about 20 nanometers to about 750 nanometers. In another preferred embodiment, the nanoparticulate contrast agent has a mean particle size of about 200 nanometers to about 400 nanometers, even more preferably about 300 nanometers to about 350 nanometers. In a particularly preferred embodiment, the nanoparticulate contrast agents have a mean particle size of less than about 300 nanometers. A nanoparticulate composition comprises a range of particle sizes. A “mean” particle size refers to the mean radius of the particles within a composition comprising a distribution of particle sizes. Particles smaller and larger than the mean size are also included in the invention. In another preferred embodiment, the nanoparticulate contrast agent is milled to achieve a particle size distribution of 50% not more than 350 nanometers and 90% not more than 1,200 nanometers.

It is to be understood that the mean particle size of the nanoparticulates used in the methods of the invention may vary depending on the desired use, e.g., mean nanoparticulate size may vary for use for local injection or administration intravascularly. It is also understood that varying the size of the nanoparticulate may increase or decrease side-effects and therefore the mean particle size may be adjusted to avoid unwanted side-effects. For example, nanoparticulates comprising a smaller mean size may result in fewer side-effects in a subject.

Nanoparticulates can be made from a broad number of materials including acrylates, methacrylates, methylmethacrylates, cyanoacrylates, acrylamides, polyacetates, polyglycolates, polyanhydrades, polyorthoesters, gelatin, polysaccharides, albumin, polystyrenes, polyvinyls, polyacroleines, polyglutataldehydes, and derivatives, copolymers, and derivatives thereof. Monomer materials particularly suitable to fabricate biodegradable nanoparticles by emulsion polymerization in a continuous aqueous phase include methylmethacrylates, polyalkycyanoacrylates, nydroxyethylmethacrylates, methacrylate acid, ethylene glycol dimethacrylate, acrylamide, N,N′-bismethyleneacrylamide and 2-dimethylaminoethyl methacrylate. Other nanoparticulates are made by different techniques from N,N-L-lysinediylterephthalate, alkycyanoacrylate, polylactic acid, polylactic acid-polyglycolic acid-copolymer, polyanhydrates, polyorthoesters, gelatin, albumin, and desolvated macromolecules or carbohydrates. Further, non-biodegradable materials can be used such as polystyrene, poly (vinylpyridine), polyacroleine and polyglutaraldehyde. A summary of materials and fabrication methods for making nanoparticulates has previously been published. See Kreuter, J. (1991) “Nanoparticles-preparation and applications.” In: M. Donbrow (Ed.) “Microcapsules and nanoparticles in medicine and pharmacy.” CRC Press, Boca Ranton, Fla., pp. 125-148.

The nanoparticulates of the invention remain substantially confined to the intravascular space and therefore do not permeate to the interstitial space or extrastitial fluids. In certain embodiments, the contrast agent is of such size to allow for phagocytosis by a mononuclear phagocyte, e.g., a macrophage, e.g., an activated macrophage. In another embodiment, the nanoparticulates of the invention are non-water soluble. In still another embodiment, the nanoparticulates of the invention comprise, or are labeleable with, a heavy element, e.g., iodine or barium, which may or may not be radioactively labeled. For example, the concentration of the heavy element, e.g., iodine, may be in a 2:1 ratio of labelable compound to iodine. In still another embodiment, the nanoparticulates of the invention have a half-life in the vasculature of a subject of at least about 30 minutes. In yet another embodiment, the nanoparticulates has a neutral pH.

The nanoparticulates suitable for use in the methods of the invention include those compositions described in, for example, U.S. Pat. Nos. 5,322,679, 5,466,440, 5,518,187, 5,580,579, and 5,718,388, the entire contents of which are hereby incorporated by reference.

In one embodiment, the nanoparticulate used in the methods of the invention is an ester of diatrizoic acid. In another embodiment, the nanoparticulate used in the methods of the invention is an iodinated aroyloxy ester. In still another embodiment, the nanoparticulate used in the methods of the invention is PH-50 (also referred to as WIN 67722 and Ni 177). PH-50 is an iodinated aroyloxy ester with the empirical formula C₁₉H₂₃I₃N₂O₆, and the chemical name 6-ethoxy-6-oxohexy-3,5-bis(acetylamino)-2,4,6-triiodobenzoate. PH-50 is cross-linked in a polymeric form and milled to generate nanoparticles and is non-soluble, e.g., non-water soluble.

In one embodiment, PH-50 formulated for use as a contrast agent comprises 150 mg/ml PH-50, 150 mg/ml polyethylene glycol 1450NF, 30 mg/ml poloxamer 338. In addition, 0.36 mg/ml tromethamine, sufficient to buffer to neutral pH, is also used. In one embodiment, the pH of PH-50 may be about 7.4.

The polymeric excipients poloxamer 338 and polyethylene glycol 1450, serve as particle stabilizers and are also intended to retard the rate of plasma clearance of particles by the reticuloendothelial system (RES) after intravascular administration. Poloxamer 338 is purified by diafiltration as a part of the manufacturing process to reduce the level of low-molecular weight polymer. Other appropriate excipients or particle stabilizers may also be used.

The physicochemical properties of the nanoparticulate of the invention are such that one would expect slow dissolution from a subcutaneous injection site providing for slow systemic absorption and metabolic attack by plasma and/or tissue esterases once the solubilized drug is absorbed. Additionally, some of the particles are transported in the lymphatics to regional lymph nodes. Mononuclear phagocyte, e.g., macrophage engulfinent of particles and subsequent phagocytosis can also occur at the injection site and within the regional lymph nodes.

In addition, in one embodiment, intravenous administration of the nanoparticulate of the invention, e.g., PH-50, results in uptake by mononuclear phagocytes, e.g., macrophages, in the reticuloendothelial system (RES), e.g., liver, spleen, or bone marrow, with subsequent intracellular dissolution and/or metabolism, and/or redistribution into plasma.

Methods of making finely milled or divided particles, e.g., nanoparticulates, of pharmaceutically active agents and drug carriers are well known in the art and the size and size range of such particles in pharmaceutical compositions can be closely controlled. For example, the nanoparticulate drug delivery vehicles used in the methods of the invention may be produced by any process known in the art for the production of the desired particle size, or by methods described in, for example, U.S. Pat. Nos. 5,718,388 and 5,518,187.

II. Methods of Use

The nanoparticulate drug delivery vehicles of the instant invention are preferably taken up by mononuclear phagocytes, e.g., macrophages. Accordingly, these nanoparticulates can serve as a means for delivering agents, e.g., pharmaceutically active agents, selectively to areas of inflamed, infected, injured, or diseased tissue or to mononuclear phagocytes e.g., macrophages, for example, areas of accumulated macrophages including diseased or inflamed tissues, or to vasculature containing plaque, lymph nodes, liver, and spleen. In one embodiment, certain pathogens, e.g., bacterial, viral, or fungal pathogens, e.g., tuberculosis, wherein the infective agent is contained within the macrophage, may be targeted with the agents of the present invention. The present invention is not limited to a particular tissue or area of the body which may be targeted for delivery and/or imaging using the methods and compositions of the invention.

Recent evidence suggests that inflammation in the vasculature, such as the coronary arteries, may be intimately involved in the development of atherosclerosis and its associated acute coronary syndromes. As a part of this inflammatory response, mononuclear phagocytes, e.g., macrophages, migrate to and accumulate at the site of plaque formation. Accordingly, one aspect of the invention provides a method of delivering pharmaceutically active agents using nanoparticulate drug delivery vehicle to accumulated mononuclear phagocytes, e.g., macrophages in a blood vessel, e.g., an artery such as a coronary or pulmonary artery, by administering, e.g., intravenously, to a subject, e.g., a mammal, such as a human, an effective amount of a nanoparticulate drug delivery vehicle so as to destroy and/or treat the vascular plaque. Furthermore, the invention includes methods for treating ischemic, inflamed, injured, or infected tissues, or vessels, vascular wall damage, and the like, using the nanoparticulate drug delivery vehicle of the invention based on the delivery of pharmaceutically active agents to areas of inflamed, infected, injured, or diseased tissue or to mononuclear phagocytes e.g., mononuclear phagocytes, e.g., macrophages, e.g., activated macrophages, at the site of ischemia, inflammation, injury, or infection. In one embodiment, the pharmaceutically active agent is a radiopharmaceutical which may destroy the target mononuclear phagocytes, e.g., macrophages, thereby treating mononuclear phagocyte-associated disorders, including vascular plaque formation, and related diseases or disorders such as tumorigenic diseases or disorders.

In another embodiment, the pharmaceutically active agents may be delivered specifically to lymph nodes to target cancer, e.g., tumors in the areas of uptake. Moreover, in a further embodiment, pharmaceutically active agents may be delivered locally to a site of inflammation, infection, or injury for treatment of mononuclear phagocyte-associated diseases or disorders, for example, inflamed joints or tissues caused by arthritis, or any other inflammatory or immune disorder. In still another embodiment, the agent may be delivered systemically in order to treat systemic or widespread inflammation, infection, injury, disease, or other tumorigenic or mononuclear phagocyte-associated diseases or disorders.

In one embodiment of the invention, the nanoparticulate drug delivery vectors are also contrast agents. The administration of nanoparticulate drug delivery agents which are formulated as contrast agents allow for the simultaneous delivery of pharmaceutically active agents and visualization, e.g., detection or imaging, of the contrast agent using any imaging techniques which are well-known in the art. These techniques may include, but are not limited to, x-ray imaging, ultrasonagraphy, computed tomography (CT), computed tomography angiography (CTA), electron beam (EBT), magnetic resonance imaging (MRI), magnetic resonance angiography (MRA), and positron emission tomography. Preferably, the detection is by CT. Imaging may be carried out at any time including prior to drug delivery, simultaneously with drug delivery, or following drug delivery.

III. Imaging Technology Used in the Methods of the Invention

As used herein, the term “imaging” or “clinical imaging” refers to the use of any imaging technology to visualize a structure, e.g., a blood vessel, e.g., a capillary, blood pool, or plaque, either in vivo or ex vivo by measuring the differences in absorption of energy transmitted by or absorbed by the tissue. Imaging technology includes x-ray technology, scanning thermography such as ultrasonagraphy, computed tomography (CT), magnetic resonance (MRI or NMR), and radionucleotides, i.e., ¹²³I or ¹²⁵I, for use in techniques such as positron emission tomography and the like.

CT imaging involves measuring the radiodensity of matter. Radiodensity is typically expressed in Hounsefield Units (HU). Hounsefield Units are a measure of the relative absorption of computed tomography X-rays by matter and is directly proportional to electron density. Water has been arbitrarily assigned a value of 0 HU, air a value of −1000 HU, and dense cortical bone a value of 1000 HU. Conventional CT scanners produce a narrow beam of x rays that passes through the subject and is picked up by a row of detectors on the opposite side. The tube and detectors are positioned on opposite sides of a ring that rotates around the patient, although the tube is unable to rotate continuously. After each rotation the scanner must stop and rotate in the opposite direction. Each rotation acquires an axial image of approximately 1 cm in thickness, at approximately 1 second per rotation. The table moves the patient a set distance through the scanner. Spiral (helical) CT scanners comprise a rotating tube, which allows a shorter scan time and more closely spaced scans. Angiography is possible with spiral scanning. Multislice CT scanners are considered “supercharged” spiral scanners. Where conventional and spiral scanners use a single row of detectors to pick up the x-ray beam, multislice scanners have up to eight active rows of detectors. Multislice scanners give faster coverage of a given volume of tissue. Various types of CT technology used in clinical practice is described in, for example, Garvey, C. and Hanlon, R. (2002) BMJ 324:1077.

In CTA, iodinated contrast agents are injected intravenously and images are obtained. Highly detailed images of the vasculature are generally obtained using CTA by reformatting the axial images to yield a composite picture of the vessels. During this reformatting, the picture of the vasculature is optimized based on the measured density in the vessels being visualized. To perform this imaging, various baseline image subtractions are performed.

CT imaging techniques which are employed are conventional and are described, for example, in Computed Body Tomography, Lee, J. K. T., Sagel, S. S., and Stanley, R. J., eds., 1983, Ravens Press, New York, N.Y., especially the first two chapters thereof entitled “Physical Principles and Instrumentation”, Ter-Pogossian, M. M., and “Techniques”, Aronberg, D. J., the disclosures of which are incorporated by reference herein in their entirety.

In one embodiment, the methods of the invention are carried out by the following procedure. A series of CT images is acquired with appropriate temporal resolution beginning just prior to contrast medium administration and continuing through the period of contrast agent administration (1-30 seconds, 1 minute, 5 minutes, 10 minutes, 15 minutes, 20 minutes, 30 minutes, 40 minutes, 50 minutes, 60 minutes, 90 minutes, 120 minutes, or more) and for a selected time period after the administration. In another embodiment, imaging is carried out after administration of the contrast agent. A wide range of image acquisition periods can be used in the method of the invention.

For example, in one embodiment, the selected time period is from about 10 seconds postcontrast to about 10 hours postcontrast, from about 30 seconds postcontrast to about 3 hours postcontrast, more preferably from about 50 seconds postcontrast to about 1 hour postcontrast, or more preferably still from about 1 minute postcontrast to about 10 minutes postcontrast. In another embodiment, the selected time period is from the time of completion of the contrast agent to about 30, 40, 50, 60 seconds postcontrast, to about 5, 10, 15, 20, 30, 40, 50, 60 minutes postcontrast, or to about 1, 2, 3, 4, 5, 6, 7, 8, 9, or more hours post contrast. Multiple images or series of images may be taken after a single administration of a contrast agent of the invention, e.g., PH-50.

A typical series might include an image every five seconds before and during the contrast medium administration, slowing further to an image every ten seconds for the subsequent three minutes, and finally slowing to an image every 30 seconds until the 10 minute completion of the series. These serial images are used to generate the dynamic enhancement data from the tissue and from the blood as measured in a vessel to be used for kinetic modeling and, ultimately, to the calculation of blood volume and perfusion within the tissue of interest. After the completion of the dynamic acquisition localized to to the region-of-interest, it may be elected to acquire additional CT images of the patient in other anatomic sites to extract additional diagnostic data or for delayed images in the same site. After CT scanning, the subject is removed from the scanner unit, and the intravenous catheter used for injection of the contrast agent can be removed. The data acquired from the CT imaging procedure is processed to provide the necessary information.

The contrast enhanced CT images can be used, for example, to define the location, caliber, and flow characteristics of vascular structures within the scanned anatomic regions as well as mononuclear phagocyte e.g., macrophage accumulation and plaque accumulation. Moreover, the images can be utilized to monitor the effect of potentially therapeutic drugs which are expected to alter perfusion status, e.g., microvascular perfusion status.

The methods described herein are useful with substantially any tissue type. In one embodiment, the tissue is a member selected from the group consisting of normal tissue, diseased tissue, and combinations thereof. In a further preferred embodiment, the tissue is at least partially a diseased tissue and the diseased tissue is a member selected from the group consisting of tissues which are neoplastic, ischemic, hyperplastic, dysplastic, inflamed, traumatized, infarcted, necrotic, infected, healing, and any combination thereof.

IV. Pharmaceutical Compositions

The nanoparticulates of the invention, e.g., PH-50, may be used as nanoparticulate drug delivery vehicles for delivery to a specific area or tissue, e.g., inflamed, infected, injured, or disease tissue or to sites containing mononuclear phagocytes e.g., macrophages, e.g., accumulated macrophages, for treatment of inflammatory diseases or disorders, infectious diseases or disorders, infected tissue, tumorigenic diseases or disorders, injured tissue or diseases tissue, e.g., mononuclear phagocyte-associated diseases or disorders.

For example, in one embodiment, the nanoparticulate may be coated by one or more pharmaceutically active agents. The resulting pharmaceutical composition may be a sustained release formulation, e.g., it may provide for delivery of a pharmaceutically active agent over an extended period. Depending on the desired drug release properties, a nanoparticulate may be coated with a single layer of coating, or alternating coatings may be provided, or the pharmaceutically active agent may actually be interdispersed within a coating material (see, e.g., U.S. Pat. No. 6,406,745 and Modem Pharmaceutics, Second Edition, edited by Gilbert S. Banker and Christopher T Rhodes, the entire contents of which is hereby incorporated by reference). Materials used for the coating include most solids currently used in the pharmaceutical and food industries, namely any material that can be effectively ablated by the energy source. These materials include, but are not limited to, biodegradable and biocompatible polymers, polysaccharides, and proteins. Suitable biodegradable polymers include polylactides, polyglycolides, polycaprolactones, polydioxanones, polycarbonates, polyhydroxybutyrates, polyalkylene oxalates, polyanhydrides, polyamides, polyesteramides, polyurethanes, polyacetates, polyketals, polyorthocarbonates, polyphosphazenes, polyhydroxyvalerates, polyalkylene succinates, poly(malic acid), poly (amino acids), polyvinylpyrrolidone, polyethylene glycol, polyhydroxycellulose, polyorthoesters, and combinations thereof, as well as other polylactic acid polymers and copolymers, polyorthoesters, and polycaprolactones, etc. Suitable biocompatible polymers include polyethyleneglycols, polyvinylpyrrolidone, and polyvinylalcohols, etc. Suitable polysaccharides include dextrans, cellulose, xantham, chitins and chitosans, etc. Suitable proteins include polylysines and other polyamines, collagen, albumin, etc. A number of materials particularly useful as coating materials are disclosed in U.S. Pat. No. 5,702,716.

In another embodiment, the nanoparticulate is a hollow sphere, semi-sphere, or liposome in which one or more pharmaceutically active agents are encapsulated for delivery, for example, for sustained release delivery.

In a further embodiment, the nanoparticulate may be conjugated, e.g., covalently or non-covalently conjugated, to one or more pharmaceutically active agents as described in, for example, U.S. Pat. No. 6,482,439, or by methods known in the art. For example, it may be desirable to directly couple a pharmaceutically active agent and a nanoparticulate or to couple a pharmaceutically active agent and a nanoparticulate via a linker group or bridging agent. More than one pharmaceutically active agent may be coupled to a polyvalent nanoparticulate.

The pharmaceutically active agent can also either be adsorbed (or absorbed) to a pre-made nanoparticulate or it can be incorporated into the nanoparticulate during the manufacturing process. Methods of absorption, adsorption, and incorporation are common knowledge to those skilled in the art.

One or more oligonucleotides may also be associated with the nanoparticulates of the invention. For example, an oligonucleotide may have a functional group associated therewith which can bind to the nanoparticles. The nanoparticulates may be, for example, positively charged.

The pharmaceutically active agent itself, e.g., a non-water soluble agent, may be formulated as a nanoparticulate for administration, e.g., by methods known in the art and described herein for preparation of nanoparticulates. In a further embodiment, the nanoparticulate drug delivery vehicle may be enzymatically degradable. Upon administration of an enzymatically degradable nanoparticulate to a subject, the nanoparticulate composition is degraded, leaving the pharmaceutically active agent.

A pharmaceutical composition of the invention, e.g., a nanoparticulate drug delivery vehicle in combination with a pharmaceutically active agent or a pharmaceutically active agent formulated as a nanoparticulate, is administered to a subject in an amount effective to allow treatment of a mononuclear phagocyte-associated disease or disorder. In one embodiment, the pharmaceutical composition of the invention may also comprise one or more pharmaceutically-acceptable carrier(s).

In a particular embodiment, the pharmaceutical composition is administered to the subject using a pharmaceutically-acceptable formulation, e.g., a pharmaceutically-acceptable formulation that suitable for administration in liquid form, including parenteral administration, for example, by intravenous injection, either as a bolus or by gradual infusion over time, intraperitoneally, intramuscularly, intracavity, subcutaneously, transdermally, dermally, by inhalation, or directed directly into the vascular tissue of interest as, for example, a sterile solution or suspension.

In certain embodiments, the subject is a mammal, e.g., a primate, e.g., a human. As used herein, the language “subject” is intended to include human and non-human animals. Preferred human animals include a human patient suffering from, or prone to suffer from, a mononuclear phagocyte-associated disease or disorder. The term “non-human animals” of the invention includes all vertebrates, e.g., mammals, e.g., rodents, e.g., mice, and non-mammals, such as non-human primates, sheep, dogs, cows, chickens, rabbits, amphibians, reptiles and the like.

The phrase “pharmaceutically acceptable” is employed herein to refer to those pharmaceutical composition of the present invention which are, within the scope of sound medical judgement, suitable for use in contact with the tissues of human beings and animals without excessive toxicity, irritation, allergic response, or other problem or complication, commensurate with a reasonable benfit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as poloxamer 338 and polyethylene glycol 1450; (9) oils, such as peanut oil, cottonseed oil, safflower oil, sesame oil, olive oil, corn oil, and soybean oil;

(10) polyols, such as glycerin, sorbitol, mannitol and polyethylene glycol; (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (18) ethyl alcohol; (19) phosphate buffer solutions; and (20) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Methods of preparing these compositions include the step of bringing into association a nanoparticulate contrast agent with the carrier and, optionally, one or more accessory ingredients. Usually, the formulations are prepared by uniformly and intimately bringing into association a contrast agent with liquid carriers.

Liquid dosage forms for oral administration of the contrast agent(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active nanoparticulate contrast agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more contrast agent(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Pharmaceutical composition of the present invention which are suitable for vaginal administration also include pessaries, tampons, creams, gels, pastes, foams or spray formulations containing such carriers as are known in the art to be appropriate.

Pharmaceutical compositions of this invention suitable for parenteral administration comprise one or more contrast agent(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, e.g., F108.

These pharmaceutical composition may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of the pharmaceutical composition, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of pharmaceutical compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the pharmaceutical composition is administered to humans and animals, it can be given per se or as a pharmaceutical composition containing, for example, 0.1 to 99.5% (more preferably, 0.5 to 90%) of active ingredient, e.g., one or more pharmaceutically active agents, in combination with a pharmaceutically-acceptable carrier.

The term “administration” or “administering” is intended to include routes of introducing the nanoparticulate drug delivery vehicle to a subject to perform their intended function. Examples of routes of administration which can be used include, for example, injection (subcutaneous, intravenous, parenterally, intraperitoneally, intrathecal). The pharmaceutical preparations are, of course, given by forms suitable for each administration route. For example, these preparations are administered, for example, by injection. The injection can be bolus or can be continuous infusion. The pharmaceutical compositions of the invention may also be administered via inhalation.

Depending on the route of administration, the pharmaceutical composition can be coated with or disposed in a selected material to protect it from natural conditions which may detrimentally effect its ability to perform its intended function. The pharmaceutical composition can be administered alone, or in conjunction with either another agent as described above or with a pharmaceutically-acceptable carrier, or both. The pharmaceutical composition can be administered prior to the administration of the other agent, simultaneously with the agent, or after the administration of the agent. Furthermore, the pharmaceutical composition can also be administered in a proform, e.g., wherein the pharmaceutically active agent is in the form of a prodrug, which is converted into its active metabolite, or more active metabolite in vivo.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically”, “peripheral administration” and “administered peripherally” as used herein mean the administration of a nanoparticulate drug delivery vehicle, drug or other material, such that it enters the patient's system and, thus, is subject to metabolism and other like processes, for example, subcutaneous administration.

Regardless of the route of administration selected, the pharmaceutical compositions of the present invention are formulated into pharmaceutically-acceptable dosage forms by conventional methods known to those of skill in the art.

To use the pharmaceutical compositions of the present invention, the pharmaceutically active agent and the nanoparticulate drug delivery vehicle are given in a dose which is pharmaceutically effective. A “pharmaceutically effective amount” or “effective amount” of a nanoparticulate drug delivery vehicle and the pharmaceutically active agent of the present invention is typically an amount such that when administered in a physiologically tolerable composition is sufficient to treat, alleviate, or prevent a mononuclear phagocyte-associated disease or disorder, within the subject. Typical dosages can be administered based on body weight, and typically are in the range of about 0.1 mL/kg to about 8.0 mL/kg, about0.2 mL/kg to about 7.0 mL/kg, about0.3 mL/kg to about 6.0 mL/kg, . about 4 mL/kg to about 5.5 mL/kg, about0.5 mL/kg to about 4.0 mL/kg, about0.6 mL/kg to about 3.5 mL/kg, about0.7 mL/kg to about 3.0 mL/kg, about0.8 mL/kg to about 2.5 mL/kg, about0.9 mL/kg to about 2.0 mL/kg, or about 1.0 mL/kg to about 1.5 mL/kg, based on a stock solution of about 150 mg/mL consisting of about 15% weight/volume [w/v]. The administration of the pharmaceutical composition of the invention may be over a period of time, e.g., by infusion, or by a single administration. In one embodiment, the administration rate of the pharmaceutical composition is about 0.6 mL/sec to about 3 mL/sec.

The dosage of the nanoparticulate drug delivery vehicle may also vary with the radioactivity of a radioisotope and will be taken into account in determining a suitable dose to be given of the contrast agent of the present invention. For example, the mean lethal dosages of both ¹²⁵I and ¹²³I have been calculated at about 79+/−9 cGy (in Chinese hamster ovary cells; see, e.g., Makrigiorgos, et al. Radiat. Res. 11:532-544). For diagnostic purposes, the dosage will be less than the mean lethal dose for the radioisotope.

For example, with respect to the half-life of common radioisotopes, the half-life of ¹²³I at a dose between 1 and 20 mCi is about 13 hours, while the half-life of ¹³¹I at a dose of less than 5 mCis about 8 days. It is expected that a useful dose of ¹²³I-labeled contrast agent would be between 1 and 20 mCi, while less than 5 mCi of the longer-lived ¹³¹I would be used (e.g. 0.5-5 mCi). Thus, for use according to the present invention, the preferred dose of agents including radioisotopes with longer half-lives will be less than the preferred dose of agents including radioisotopes with shorter half-lives.

Compositions comprising the pharmaceutical composition are conventionally administered intravenously, as by injection of a unit dose, for example. The term “unit dose” when used in reference to a pharmaceutical composition of the present invention refers to physically discrete units suitable as unitary dosage for the subject, each unit containing a predetermined quantity of active material calculated to produce the desired effect in association with the required diluent, i.e., carrier. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a desired effect. Generally, out of one hundred percent, this amount will range from about 1 percent to about ninety-nine percent of active ingredient, preferably from about 5 percent to about 70 percent, most preferably from about 10 percent to about 30 percent.

The nanoparticulate drug delivery vehicle and the pharmaceutically active agents are administered in a manner compatible with the dosage formulation, and in an effective amount. The quantity to be administered depends on the subject, capacity of the subject's system to utilize the active ingredient, the degree of contrast desired, and the structure to be imaged. Precise amounts of the drug delivery vehicle and the pharmaceutically active agents required to be administered depend on the judgement of the practitioner and are peculiar to each individual. However, suitable dosage ranges for systemic application are disclosed herein and depend on the route of administration. Suitable regimes for initial administration and subsequent administration, e.g., after initial imaging, are also contemplated and are typified by an initial administration followed by repeated doses at one or more hour intervals by a subsequent injection or other administration. Bolus administration, multiple dosages or continuous intravenous infusion sufficient to maintain concentrations in the blood in the ranges for specific in vivo imaging are also contemplated. Infusion of the contrast agent may be for less than one minute, two minutes, three minutes, four minutes, five minutes, or more.

V. Kits

It is anticipated that the methods and the pharmaceutical compositions of the invention can be incorporated into a commercial kit or system for treating mononuclear phagocyte-associated diseases or disorders and/or imaging, detecting, and evaluating the perfusion and extravasation of blood out of vascular tissue, including but not limited to, vascular beds (e.g., arterial and venous beds), organ tissues (e.g., myocardial tissues and other organ tissues), and tumors, e.g., for the measurement of angiogenesis or perfusion status of tumors, or for the imaging, detecting, and evaluating mononuclear phagocyte e.g., macrophage accumulation or plaque accumulation. The kit may contain a nanoparticulate drug delivery vehicle, pharmaceutically active agent, and instructions for use and may further contain directions on the administration and use of the nanoparticulate drug delivery vehicle and/or pharmaceutically active agent, and dosage requirement for the intended use.

Other features, advantages and embodiments of the invention will be apparent from the following examples which are meant to illustrative, and therefore, not limiting in any way.

### EXAMPLES

### Example 1

### Example of Nanoparticulate Used in the Methods of the Invention:

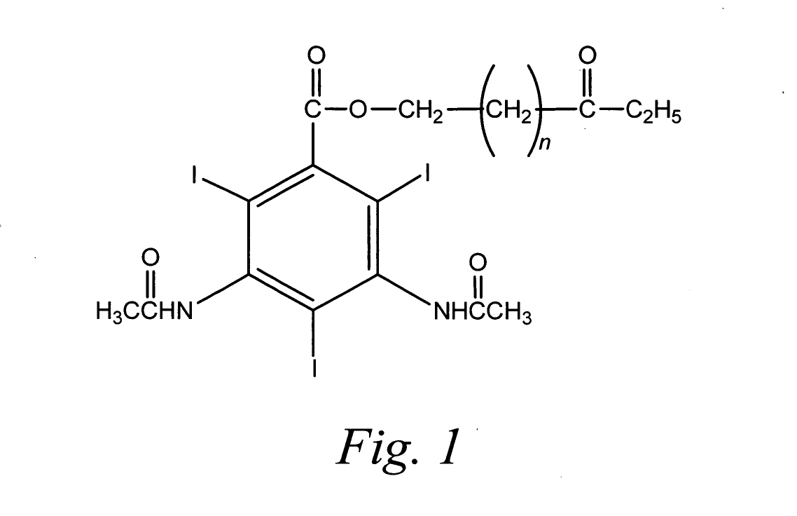

Sterile WIN 67722 Suspension 150 mg/mL (referred to herein as “Sterile PH-50”, “PH-50 Injectable Suspension” or “PH-50 drug product”) is a parenteral iodinated x-ray contrast agent which has been utilized for indirect lymphography. The PH-50 compound is described, for example, in U.S. Pat. Nos. 5,322,679, 5,466,440, 5,518,187, 5,580,579, and 5,718,388. PH-50 has the empirical formula C₁₉H₂₃I₃N₂O₆ and has the chemical name 6-ethoxy-6-oxohexy-3,5-bis(acetylamino)-2,4,6-triiodobenzoate, an esterified derivative of the x ray contrast agent diatriazoic acid. PH-50 has a molecular weight of 756.1. The structural formula for PH-50 is shown in FIG. 1. The PH50 compound can be produced by the condensation of ethyl 6-bromohexanoate with sodium diatrizoate in DMF followed by the precipitation of the product from DMSO and washing with ethanol. PH-50 can be obtained from Sigma-Aldrich Fine Chemicals.

The concentration of iodine in PH-50 Injectable Suspension is 76 mg/mL. PH-50 Injectable Suspension is a white to off-white crystalline material containing 50.35% iodine (by weight), and has a low water solubility (<10 μg/mL).

The PH-50 drug product is milled to achieve a particle size distribution of 50% not more than about 350 nanometers and 90% not more than about 1,200 nanometers. The milled dug product can be obtained from Nanosystems, Inc.

The final formulation of PH-50 Injectable Suspension is as set forth in Table 1, below:

TABLE 1
PH 50 Formulation

| | MW | Molar Conc. | Mass Conc. |
|---|---|---|---|
| Component | (g/mole) | (M) | (mg/ml) |
| PH-50 | 756.12 | 0.198 | 150 |
| Polyethylene glycol 1450 NF | ˜15,000 | 0.01 | 150 |
| Poloxamer 338 | ˜14,760 | 0.002 | 30 |
| Tromethamine-sufficient | 121.14 | 2.97 | 0.36 |
| to buffer to neutral pH | | | |

Relevant Formulation Specifications:
pH ˜7.4
Particle Size 50% NMT 350 nm
90% NMT 1200 nm

The polymeric excipients, poloxamer 338 and polyethylene glycol 1450, serve as particle stabilizers and are also intended to retard the rate of plasma clearance of particles by the reticuloendothelial system (RES) after intravascular administration. Poloxamer 338 is purchased from BASF®, and is purified by diafiltration as a part of the manufacturing process to reduce the level of low-molecular weight polymer.

The physicochemical properties of the drug particles are such that one would expect slow dissolution from a subcutaneous injection site providing for slow systemic absorption and metabolic attack by plasma and/or tissue esterases once the solubilized drug is absorbed. Additionally, some of the particles are transported in the lymphatics to regional lymph nodes. Macrophage engulfinent of particles and subsequent phagocytosis can also occur at the injection site and within the regional lymph nodes. In addition, IV administration of PH-50 should result in uptake by macrophages in the RES, e.g., liver, spleen, bone marrow) with subsequent intracellular dissolution and/or metabolism or redistribution into plasma.

### Example 2

### Delivery of Nanoparticulates Used in the Methods of the Invention:

The following are examples showing PH-50 taken up by macrophages in the desired areas of treatment.

Following intravenous injection of PH-50 in rabbits, computer tomography images were taken at different time points after injection.

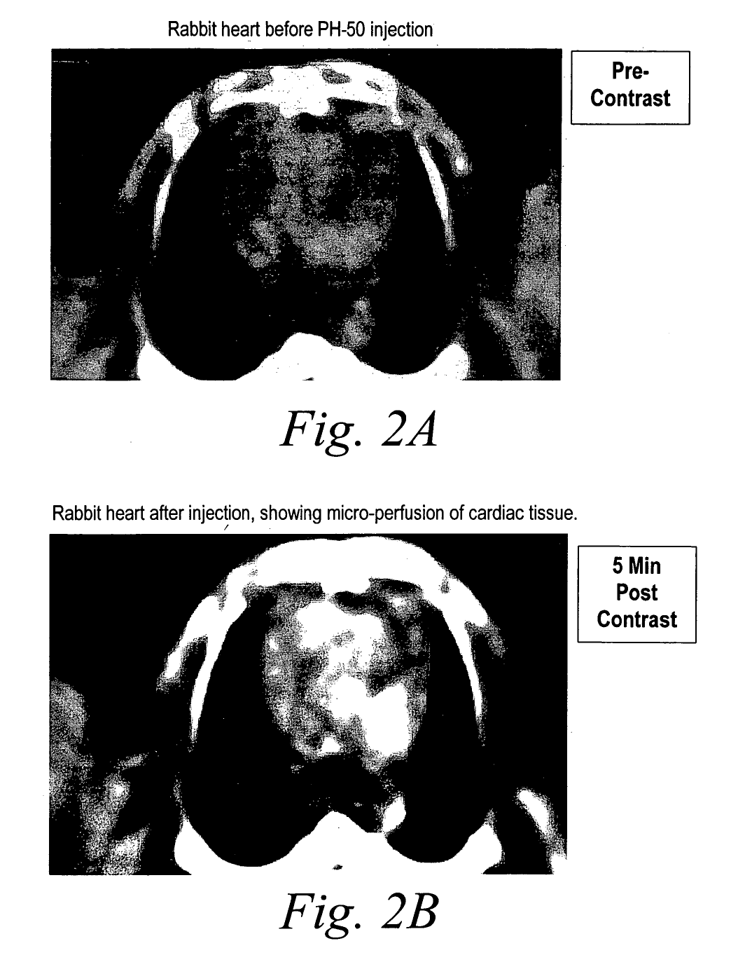

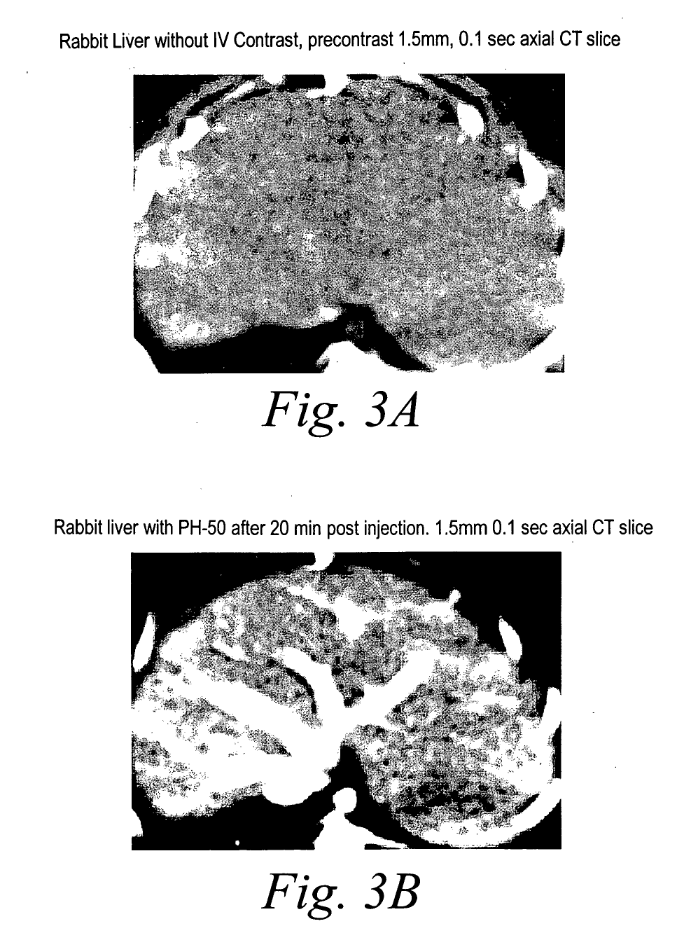

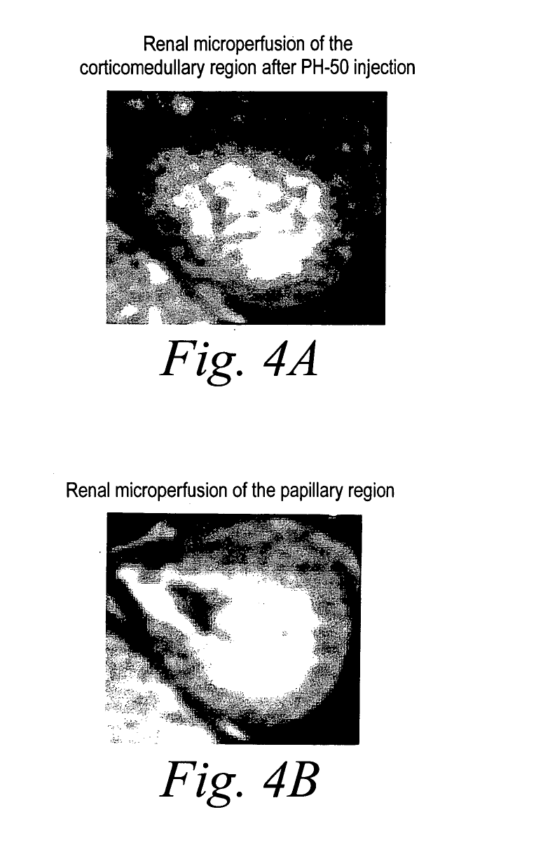

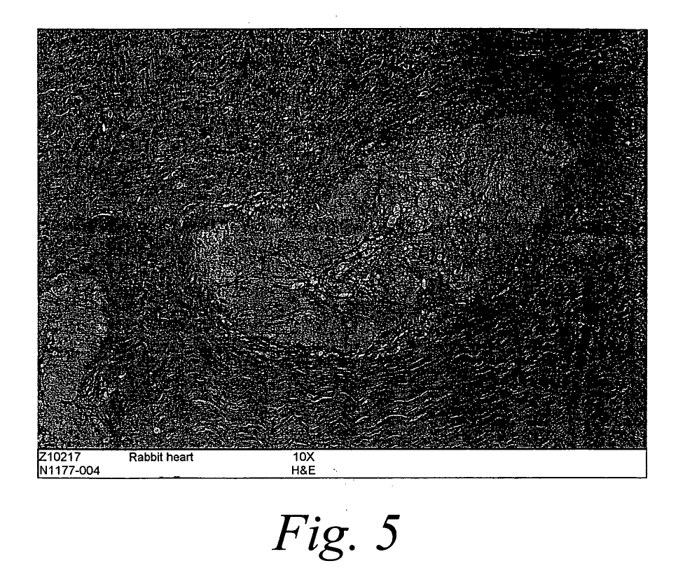

For example, FIGS. 2A and 2B show a CT scan of a rabbit heart 5 minutes prior to PH-50 injection (FIG. 2A) and 5 minutes after PH-50 injection (FIG. 2B). FIG. 3 shows the micro-perfusion of the cardiac tissue in the rabbit heart. FIGS. 3A and 3B illustrate the effectiveness of PH-50 in a rabbit liver. In particular, FIG. 3A shows a CT scan of a rabbit liver prior to PH-50 injection. FIG. 3B shows the rabbit liver 20 minutes post PH-50 injection wherein the microperfusions in the tissue are clearly illuminated. FIGS. 4A and 4B illustrate the results of scanning various aspects of a rabbit kidney after PH-50 injection. Each of these figures demonstrate the ability of the nanoparticulates to be taken up by a particular organ. Hence, a pharmaceutically active compound associated with the nanoparticulate would also be taken up by that organ or tissue and delivered thereto for treatment.

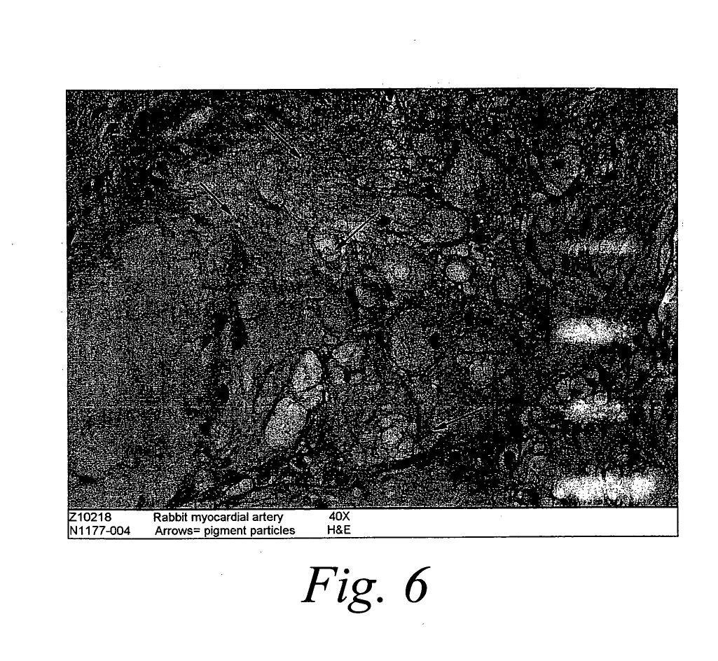

FIGS. 5 and 6 illustrate where nanoparticulates are taken up in vascular plaque, as explained below in Table 2:

TABLE 2
LIGHT MICROSCOPY PHOTO LEGEND

| Photo | | | | |
|---|---|---|---|---|
| Number/ | Animal | | | |
| FIG. | Number | Objective | Stain | Comment |
| Z10217/ | 3086 | 1Ox | Hematoxylin & | Rabbit heart with |
| | | | Eosin | atherosclerotic |
| | | | | plaques. |
| Z102187 | 3086 | 40x | Hematoxylin & | Pinpoint pigment |
| | | | Eosin | granules (arrows) |
| | | | | are located in the |
| | | | | atherosclerotic plaque |
| | | | | of a rabbit |
| | | | | myocardial artery. |

Hence, FIGS. 5 and 6 demonstrate that the nanoparticulates can be delivered specifically to a certain area and be taken up by the macrophages in that particular area. The use of other, non-iodinated nanoparticulate contrast agents for imaging of microperfusion of vascular tissues as well as non-contrast agents that have been formulated as nanoparticulates for the purpose of drug-delivery fall within the scope of the present invention. For example, use of other nanoparticulate x-ray contrast agents containing various other heavy elements, such as barium, with x-ray-based imaging, is within the scope of the present invention. Furthermore, use of various nanoparticulate agents with other imaging modalities, such as magnetic resonance imaging (MRI) and ultrasound, is within the scope of the present invention.

Incorporation By Reference

The contents of all references (including literature references, issued patents, published patent applications, and co-pending patent applications) cited throughout this application are hereby expressly incorporated herein in their entireties by reference.

Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. A pharmaceutical composition comprising a nanoparticulate drug delivery vehicle and a pharmaceutically active agent.

2. The pharmaceutical composition of claim 1, wherein said nanoparticulate drug delivery vehicle is non-water soluble.

3. The pharmaceutical composition of claim 1, wherein said nanoparticulate drug delivery vehicle is PH-50.

4. The pharmaceutical composition of claim 1, wherein the mean particle size of said nanoparticulate drug delivery vehicle is from about 20 nanometers to about 750 nanometers.

5. The pharmaceutical composition of claim 1, wherein the mean particle size of said nanoparticulate drug delivery vehicle is from about 200 nanometers to about 400 nanometers.

6. The pharmaceutical composition of claim 1, wherein the mean particle size of said nanoparticulate drug delivery vehicle is about 300 nanometers.

7. The pharmaceutical composition of claim 1, wherein said nanoparticulate drug delivery vehicle is a contrast agent.

8. The pharmaceutical composition of claim 1, wherein said nanoparticulate drug delivery vehicle is enzymatically degradable.

9. The pharmaceutical composition of claim 1, wherein the mean particle size of said nanoparticulate drug delivery vehicle is of a size sufficient to be taken up by a mononuclear phagocyte.

10. The pharmaceutical composition of claim 9, wherein said mononuclear phagocyte is a macrophage.

11. The pharmaceutical composition of claim 1, wherein said nanoparticulate drug delivery vehicle and said pharmaceutically active agent are covalently conjugated.

12. The pharmaceutical composition of claim 1, wherein said pharmaceutically active agent coats the surface of said nanoparticulate drug delivery vehicle.

13. The pharmaceutical composition of claim 1, wherein said nanoparticulate drug delivery vehicle contains a hollow core and wherein said pharmaceutically active agent is encapsulated by said nanoparticulate.

14. The pharmaceutical composition of claim 1, wherein said pharmaceutically active substance is a prodrug.

15. The pharmaceutical composition of claim 1, wherein said prodrug is formulated as a nanoparticulate.

16. The pharmaceutical composition of claim 15, wherein said prodrug is converted to an active substance upon uptake by a mononuclear phagocyte.

17. The method of claim 1, wherein said pharmaceutically active agent is a radiopharmaceutical agent.

18. A method for treatment of an inflammatory disease or disorder or infectious disease or disorder in a subject comprising administering to said subject an effective amount of a pharmaceutical composition comprising a nanoparticulate drug delivery vehicle and a pharmaceutically active agent, thereby treating said inflammatory disease or disorder or infectious disease or disorder.

19. The method of claim 18, wherein said nanoparticulate drug delivery vehicle is non-water soluble.

20. The method of claim 18, wherein said nanoparticulate drug delivery vehicle is PH-50.

21. The method of claim 18, wherein the mean particle size of said nanoparticulate drug delivery vehicle is from about 20 nanometers to about 750 nanometers.

22. The method of claim 18, wherein the mean particle size of said nanoparticulate drug delivery vehicle is from about 200 nanometers to about 400 nanometers.

23. The method of claim 18, wherein the mean particle size of said nanoparticulate drug delivery vehicle is about 300 nanometers.

24. The method of claim 18, wherein said nanoparticulate drug delivery vehicle is a contrast agent.

25. The method of claim 18, wherein said nanoparticulate drug delivery vehicle is enzymatically degradable.

26. The method of claim 18, wherein the mean particle size of said nanoparticulate drug delivery vehicle is of a size sufficient to be taken up by a mononuclear phagocyte.

27. The method of claim 26, wherein said mononuclear phagocyte is a macrophage.

28. The method of claim 18, wherein said nanoparticulate drug delivery vehicle is coated with said pharmaceutically active agent.

29. The method of claim 18, wherein said nanoparticulate drug delivery vehicle and said pharmaceutically active agent are covalently conjugated.

30. The method of claim 18, wherein said pharmaceutically active agent is a radiopharmaceutical agent.

31. The method of claim 18, wherein said nanoparticulate drug delivery vehicle contains a hollow core and wherein said pharmaceutically active agent is encapsulated by said nanoparticulate.

32. The method of claim 18, wherein said pharmaceutically active substance is a prodrug.

33. The method of claim 32, wherein said prodrug is formulated as a nanoparticulate.

34. The method of claim 32, wherein said prodrug is converted to an active substance upon uptake by a mononuclear phagocyte.

35. The method of claim 18, wherein said composition is administered intravenously.

36. The method of claim 18, wherein said composition is locally injected at a site of infection or inflammation.

37. The method of claim 18, wherein said pharmaceutically active agent is a sustained release agent.

38. A method for treating or removing plaque accumulation in a blood vessel of a subject comprising administering to said subject an effective amount of a composition comprising a nanoparticulate drug delivery vehicle and a pharmaceutically active agent, thereby treating or removing plaque accumulation in a blood vessel of a subject.

39. A method for treating a tumorigenic disease or disorder in a subject comprising administering to said subject an effective amount of a composition comprising a nanoparticulate drug delivery vehicle and a pharmaceutically active agent, thereby treating said tumorigenic disease or disorder.
